(19) United States
(12) Patent Application Publication
(10) Pub. No.: US 2004/0254555 A1
(43) Pub. Date: Dec. 16, 2004

(54) Absorbent articles having a heterogeneous absorbent core for fecal fluid and urine containment

(76) Inventors: James Hongxue Wang, Appleton, WI (US); Russell Paul George, Appleton, WI (US); Debra Jean McDowall, Neenah, WI (US); Kusum Gosain, Appleton, WI (US); Sharon Rymer, Neenah, WI (US); TaraLea Bastian, Waupaca, WI (US)

Correspondence Address: Maxwell J. Petersen, Pauley Petersen Kinne & Erickson, Suite 365, 2800 West Higgins Road, Hoffman Estates, IL, 60195, US

(21) Appl. No.: 10/458,396

(22) Filed: Jun. 10, 2003

(51) Int. Cl. A61F 13/15; A61F 13/20

(52) U.S. Cl. 604/385.01; 604/367

(57) ABSTRACT

A heterogeneous absorbent core for use in absorbent articles includes a first zone optimized for absorbency of urine and a second zone optimized for absorbency of fecal fluids. The first zone is closer to the front of the core in the article, and includes a first superabsorbent material having a saline absorbency under load of at least about 20 grams/gram. The second zone is closer to the back of the core in the absorbent article, and includes a second superabsorbent material having a fecal fluid absorbency under load of at least about 15 grams/gram. A diaper, training pant, swim wear article and adult incontinence garment which incorporates the heterogeneous core are also provided.

### FIELD OF THE INVENTION

This invention relates to an absorbent article having a heterogeneous absorbent core. At least one region of the absorbent core, such as the front region, is designed for optimal absorption of urine. At least one other region of the absorbent core, such as the back region, is designed for optimal absorption of viscous fecal fluid.

### BACKGROUND OF THE INVENTION

Personal care absorbent articles, such as diapers, training pants, and adult incontinence garments typically include a liquid pervious top layer (often referred to as a bodyside liner), a liquid impermeable bottom layer (often referred to as an outer cover), and an absorbent core between them. The absorbent core is often defined as including a front region (closer to the front waist of the wearer), a back region (closer to the rear waist of the wearer), and a crotch region (the lowermost region on a wearer, connecting the front region to the back region). For purposes of this document, the front region of the absorbent core may be defined as including one-third of the length of the absorbent core measured from the edge of the absorbent core which is closest to the front waist edge of the garment. The back region of the absorbent core may be defined as including one-third of the length of the absorbent core measured from the edge of the absorbent core which is closest to the rear waist edge of the garment. The crotch region of the absorbent core may be defined as including the remaining one-third of the length of the absorbent core which is bounded by the front region and the back region.

Conventional absorbent core materials include a matrix of cellulose fluff or pulp having some absorbency, and a superabsorbent material having higher absorbency dispersed within the matrix. Conventional absorbent core materials are designed for optimal absorption of urine. With this in mind, it is known to provide absorbent cores which are thicker in the front and/or crotch regions than in the back region, because most of the liquid is absorbed in the front and crotch regions. It is also known to provide absorbent cores having a higher concentration of superabsorbent material in the front and/or crotch regions than in the back region. Because the structure of the absorbent cores is optimized primarily based on the distribution of urine concentration, the type of cellulose fluff or pulp and the type of superabsorbent material are generally the same in the front, crotch and back regions of the absorbent core. Only the amounts of each are varied.

In addition to urine, absorbent articles are also subjected to insults of runny fecal matter. While the cellulose fluff or pulp and the superabsorbent material will absorb some of the runny fecal matter, this absorption is not optimum because the conventional absorbent core is not optimized for this purpose. Efforts have been made to contain runny fecal matter as well as solid fecal matter using physical barriers such as side leak guards or flaps, and grid-like features which hinder lateral movement and flow. These devices provide physical hindrance but not improved absorption. Consequently, runny fecal matter has a greater tendency to leak, than does urine, from conventional absorbent articles.

### SUMMARY OF THE INVENTION

This application is directed to an absorbent article including a liquid-permeable bodyside liner, an outer cover, and a heterogeneous absorbent core in between them. The heterogeneous absorbent core includes a first zone including a first superabsorbent material. The first superabsorbent material has an absorbency under load for 0.9% by weight aqueous saline solution at a pressure of 0.3 psi (herein “a saline AUL”) of at least about 15 grams saline solution per gram of superabsorbent material. The absorbent core also includes a second zone including a second superabsorbent material, different from the first. The second superabsorbent material has an absorbency under load for synthetic fecal fluid at a pressure of 0.3 psi (herein “a fecal fluid AUL”) of at least about 15 grams fecal fluid per gram of superabsorbent material. The second superabsorbent material may also have a saline AUL of at least about 15 grams/gram.

The first zone is located in at least part of the front region and/or at least part of the crotch region of the absorbent core. The second zone is located in at least part of the back region and/or at least part of the crotch region of the absorbent core. The second zone is located behind the first zone, i.e., closer to the rear waist edge of the absorbent core than the first zone. The first and second zones together may constitute the entire absorbent core, or may constitute less than the entire absorbent core.

This application is also directed to an absorbent article including a liquid-permeable bodyside liner, an outer cover, and a bi-functional absorbent core in between them. The bi-functional absorbent core includes a layer which includes a first superabsorbent material selected based on its high absorption of urine. The first superabsorbent material has an absorbency under load for aqueous sodium chloride at a pressure of 0.3 psi (herein “a saline AUL”) of at least about 15 grams saline solution per gram of superabsorbent material. The absorbent core includes a layer (the same or different layer) which includes a second superabsorbent material, different from the first, selected for high fecal fluid absorption. The second superabsorbent material has an absorbency under load for synthetic fecal fluid at a pressure of 0.3 psi (herein “a fecal fluid AUL”) of at least about 15 grams fecal fluid per gram of superabsorbent material. The second superabsorbent material may also have a saline AUL of at least about 15 grams/gram.

The layer(s) which include the first and second superabsorbent materials are present throughout the length of at least the crotch region of the absorbent core, and may extend lengthwise beyond the crotch region through part or all of the front region and/or part or all of the back region. Where the first and second superabsorbents are in different layers, the layers are superimposed and coextensive at least through the length of the crotch region, suitably through part or all of the front and/or back regions. The bi-functional core may or may not include additional layer(s) which do not include any of the first or second superabsorbent material.

With the foregoing in mind, it is a feature and advantage to provide a heterogeneous absorbent core having a first zone designed for optimal absorption of urine and a second zone designed for optimal absorption of fecal fluid. It is also a feature and advantage to provide an absorbent article such as a diaper, diaper pant, swim wear or adult incontinence garment, which embodies the heterogeneous absorbent core.

It is also a feature and advantage to provide a bi-functional absorbent core having a layer designed for optimal absorption of urine and a (same or different) layer designed for optimal absorption of fecal fluid. It is also a feature and advantage to provide an absorbent article such as a diaper, diaper pant, swim wear or adult incontinence garment, which embodies the bi-functional absorbent core.

### BRIEF DESCRIPTION OF THE DRAWINGS

FIG. 1 is an exploded view of an absorbent article which embodies the heterogeneous absorbent core having first and second zones according to the invention.

FIGS. 2-5 are plan views of different embodiments of a heterogeneous absorbent core having first and second zones according to the invention.

FIGS. 6-9 are perspective views of different embodiments of a bi-functional absorbent core having layer(s) optimized for urine and fecal fluid absorption.

FIGS. 10 and 11 illustrate improved embodiments of the absorbent core of FIG. 2, further including a relatively depressed region surrounded by a relatively raised region.

FIGS. 12(a) and 12(b) illustrate an apparatus used for the saline and fecal fluid AUL tests described herein.

FIG. 13 is an exploded view of an apparatus used for the CRC screen test described herein.

### DEFINITIONS

The term “absorbent matrix material” refers to materials such as cellulose fibers which are capable of absorbing at least five times but generally less than 15 times their own weight in synthetic urine using the saline AUL test described herein. The absorbent matrix material can also include synthetic fiber matrices such as spunbond, meltblown and bonded carded webs, and the like. Also included are open structures such as through-air bonded carded webs, lofty through-air bonded bicomponent fiber spunbond webs, and other materials useful for rapid fluid intake.

The term “superabsorbent material” refers to water-swellable organic and inorganic materials that are capable of absorbing at least 15 times their own weight in a solution of 0.9% by weight aqueous sodium chloride using the saline AUL test described herein and/or that are capable of absorbing at least 15 times their own weight in synthetic fecal fluid using the fecal fluid AUL test described herein.

The term “personal care absorbent article” includes without limitation diapers, training pants, swim wear, absorbent underpants, adult incontinence products, tissues, wet wipes, bed mats, and feminine hygiene articles.

The term “polymer” includes, but is not limited to, homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to isotactic, syndiotactic and atactic tacticities.

The term “heterogeneous” absorbent core includes absorbent cores having a first zone and a second zone, in which a first superabsorbent material is included only in the first zone and a second superabsorbent material is included only in the second zone. The term also includes all other absorbent cores in which the first and second superabsorbent materials are present in the first and second zones, in substantially different ratios based on the total weights of superabsorbent materials in the zones. For the ratios to be substantially different, they must differ (plus or minus) by at least about 10%, suitably by at least about 25%, particularly by at least about 50%.

By way of example, if a first zone includes a superabsorbent A₁ as the only superabsorbent material, and the second zone includes superabsorbents A₁ and A₂ in equal quantities, then the difference in ratios would be:

$${( \frac{{A}_{1}}{{A}_{1} + {A}_{2}} )}_{First Zone} {( \frac{{A}_{1}}{{A}_{1} + {A}_{2}} )}_{Second Zone} \\ = 1.0 - 0.5 = 0.5 , or 50 \%$$

If the first zone includes four parts by weight of A₁ for every six parts by weight A₂ and the second zone includes six parts by weight A₁ for every four parts by weight A₂, then the difference in ratios would be:

$${( \frac{{A}_{1}}{{A}_{1} + {A}_{2}} )}_{First Zone} {( \frac{{A}_{1}}{{A}_{1} + {A}_{2}} )}_{Second Zone} \\ = 0.4 - 0.6 = - 0.2 , or 20 \%$$

If three or more superabsorbent materials are included in either or both zones, the absorbent core is “heterogeneous” if the above test for “substantially different ratios” is passed for at least two of the superabsorbent components. For instance, if a first zone includes five parts by weight of superabsorbent A₁ for every three parts by weight of superabsorbent A₂ and two parts by weight of superabsorbent A₃, and a second zone includes five parts of A₁ for every one part of A₂ and four parts of A₃, the “substantially different ratio” test would be passed for both A₂ and A₃.

$${( \frac{{A}_{2}}{{A}_{1} + {A}_{2} + {A}_{3}} )}_{First Zone} {( \frac{{A}_{2}}{{A}_{1} + {A}_{2} + {A}_{3}} )}_{Second Zone} \\ = 0.3 - 0.1 = 0.2 , or 20 \%$$

$${( \frac{{A}_{3}}{{A}_{1} + {A}_{2} + {A}_{3}} )}_{First Zone} {( \frac{{A}_{3}}{{A}_{1} + {A}_{2} + {A}_{3}} )}_{Second Zone} \\ = 0.2 - 0.4 = - 0.2 , or 20 \%$$

In the above example, the absorbent core would be “heterogeneous” even though the weight ratio for A₁ based on total superabsorbent content is the same in both zones.

The term “bi-functional absorbent core” refers to an absorbent core having a layer which includes a first superabsorbent material selected based on its high absorption of urine and a (same or different) layer which includes a second superabsorbent material, different from the first, selected based on its high absorption of fecal fluid.

### DETAILED DESCRIPTION OF THE

### PRESENTLY PREFERRED EMBODIMENTS

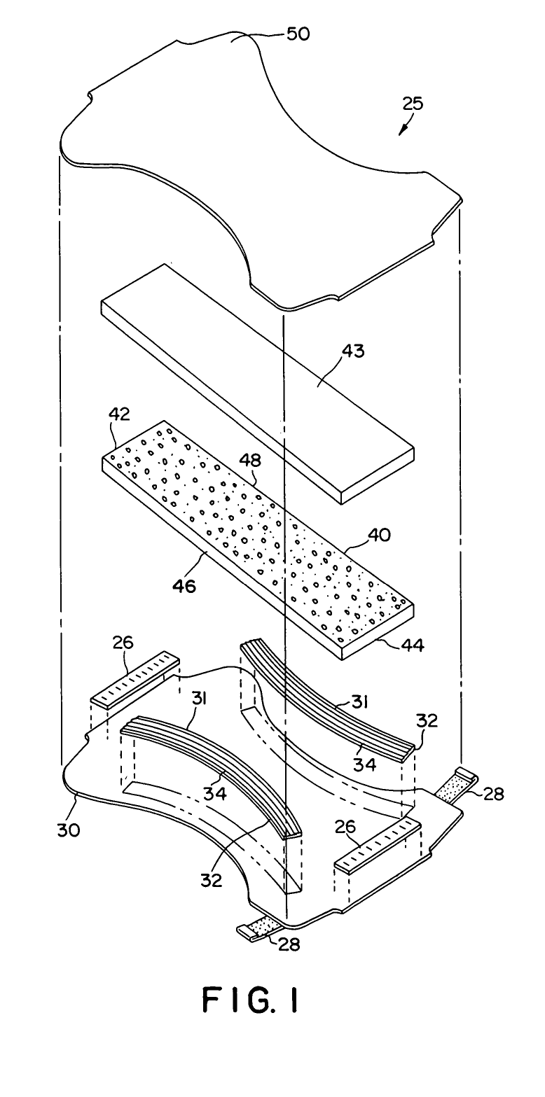

FIG. 1 illustrates a personal care article 25 according to the invention, in this case a disposable diaper. Disposable diaper 25 includes a liquid permeable bodyside liner 50, a surge material layer 43, an absorbent core 40, and an outer cover 30. The illustrated absorbent core 40 has a rectangular shape, and includes a front waist edge 42, a rear waist edge 44, and two longitudinal side edges 46 and 48. For purposes of the invention, the absorbent core 40 is not limited to a rectangular shape, and may have an hour glass shape, or another suitable shape. The surge layer and other layers can also have different shapes and dimensions.

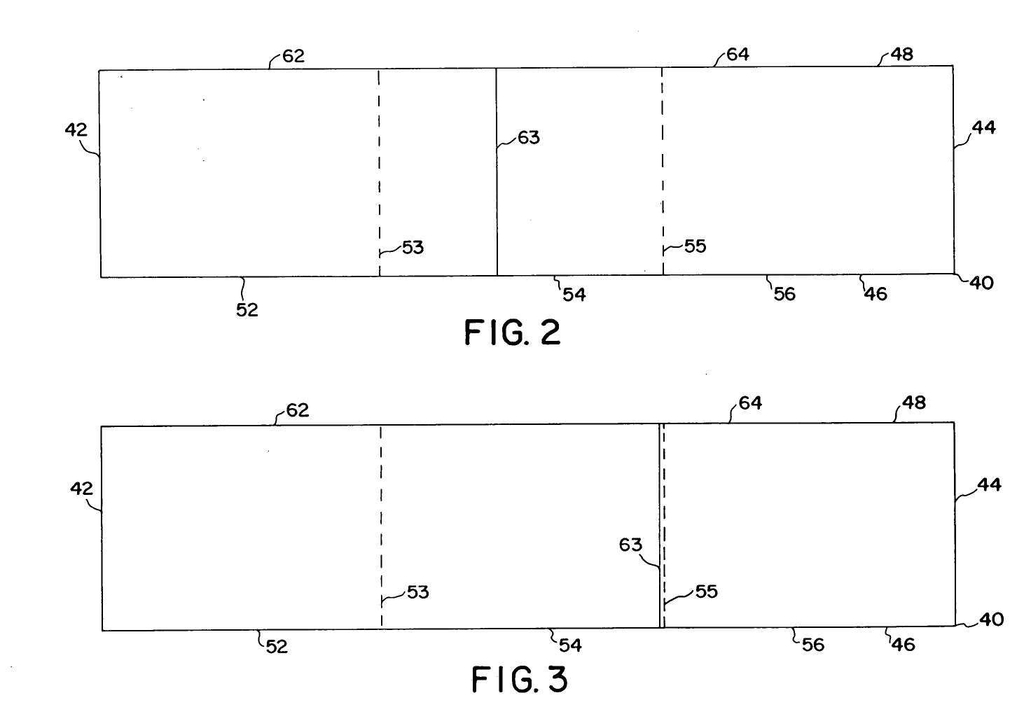

Referring to FIG. 2, absorbent core 40 includes a front region 52, a crotch region 54 and a back region 56. The regions 52, 54 and 56 are defined as each including one-third of the total length of the absorbent core 40. The front region 52 commences at the front waist edge 42 of the core 40, and includes the front one-third of the core 40 ending at an imaginary boundary line, shown as imaginary dashed line 53. The back region 56 extends from the rear waist edge 44 of the core 40, and includes the back one-third of the core bounded by the imaginary dashed line 55. The crotch region 54 includes the middle one-third of the length of the core, between the imaginary dashed lines 53 and 55.

The heterogeneous absorbent core 40 includes a first zone 62 and a second zone 64, separated by a boundary line 63. The first zone 62 includes the front region 52 and part of the crotch region 54 of the core 40. The second zone 64 includes the back region 56 and part of the crotch region 54 of the core 40.

The first zone 62 includes a first superabsorbent material having a saline AUL of at least about 15 grams 0.9% by weight aqueous sodium chloride solution per gram of superabsorbent, particularly at least about 25 grams/gram, particularly at least about 30 grams/gram at a pressure of 0.3 psi, using the saline absorbency under load test described herein. The first superabsorbent material may be dispersed in a cellulose fiber matrix, such as a matrix of wood pulp fluff or fibers, or may be dispersed in another absorbent matrix. The dispersed superabsorbent material may be in the form of particles, fibers or an in-situ-polymerized superabsorbent. Alternatively, the first superabsorbent material may be in the form of a thin film, foam or other layer. The first superabsorbent material may constitute about 10% to 100% by weight of the first zone 62, particularly about 30% to 100% by weight, particularly about 40% to 100% by weight. When an absorbent fibrous matrix is employed, the first superabsorbent material may constitute about 10% to about 90% by weight of the first zone 62, particularly about 30% to about 80% by weight, more particularly about 40% to about 70% by weight. The absorbent fibrous matrix may constitute some or all of the balance of the weight of the first zone 62. All percentages are based on the dry weights of the ingredients.

The second zone 64 includes a second superabsorbent material, different from the first, which has a fecal fluid AUL of at least about 15 grams fecal fluid per gram of superabsorbent material, particularly at least about 20 grams/gram, particularly at least about 25 grams/gram at a pressure of 0.3 psi, using the fecal fluid absorbency under load test described herein. The second superabsorbent material may also have a saline AUL of at least about 15 grams/gram, particularly at least about 25 grams/gram, particularly at least about 30 grams/gram, using the saline absorbency under load test described herein. The second superabsorbent material may be dispersed in a cellulose fiber matrix, such as a matrix of wood pulp fluff or fibers, or may be dispersed in another absorbent matrix. The dispersed superabsorbent material may be in the form of particles or fibers. Alternatively, the second superabsorbent material may be in the form of a thin film, foam or other layer. The second superabsorbent material may constitute about 10% to 100% by weight of the second zone 64, particularly about 30% to 100% by weight, particularly about 40% to 100% by weight. When an absorbent fibrous matrix is employed, the second superabsorbent material may constitute about 10% to about 90% by weight of the second zone 64, particularly about 30% to about 80% by weight, more particularly about 40% to about 70% by weight. The absorbent fibrous matrix may constitute some or all of the balance of the weight of the second zone 64. Again, all percentages are based on the dry weights of the ingredients.

As explained above, the first superabsorbent material in the first zone 62 and the second superabsorbent material in the second zone 64 may be combined with other superabsorbent materials and other ingredients, provided that the test for “substantially different ratios” defining a heterogeneous absorbent core has been satisfied. For the “substantially different ratios” test to be satisfied, at least two of the superabsorbent materials in a system containing two or more superabsorbents must exhibit a difference in ratios of at least about 10%, suitably at least about 25%, particularly at least about 50% between the first and second zones 62 and 64 of heterogeneous absorbent core 40.

The superabsorbents may be prepared from polymerizable, unsaturated, acid-containing monomers. Such monomers include the olefinically unsaturated acids and anhydrides which contain at least one carbon to carbon olefinic double bond. More specifically, these monomers can be selected from olefinically unsaturated carboxylic acids and acid anhydrides, olefinically unsaturated phosphoric acids or sulfonic acids and mixtures thereof.

Olefinically unsaturated carboxylic acid and carboxylic acid anhydride monomers include the acrylic acids such as acrylic acid, methacrylic acid, ethacrylic acid, alpha-chloroacrylic acid, alpha-cyano acrylic acid, beta-methyl acrylic acid (crotonic acid), alpha-phenyl acrylic acid, beta-acryloxy propionic acid, sorbic acid, alpha-chloro sorbic acid, angelic acid, cinnamic acid, para-chloro cinnamic acid, beta-steryl acrylic acid, itaconic acid, citraconic acid, mesaconic acid, glutaconic acid, aconitic acid, maleic acid, fumaric acid, tricarboxyethylene and maleic acid anhydride.

Olefinically unsaturated sulfonic acid monomers include aliphatic or aromatic vinyl sulfonic acids such as vinylsulfonic acid, allyl sulfonic acid, vinyltoluene sulfonic acid and styrene sulfonic acid; acrylic and methacrylic sulfonic acid such as sulfoethyl acrylate, sulfoethyl, methacrylate, sulfopropyl acrylate, sulfopropyl methacrylate, 2-hydroxy-3-acryloxy propyl sulfonic acid, 2-hydroxy-3-methacryloxy propyl sulfonic acid and 2-acrylamido-2-methyl propane sulfonic acid.

Suitable superabsorbents for use in the present invention may possess a carboxyl group. The superabsorbents include hydrolyzed starch-acrylonitrile graft copolymer, partially neutralized starch-acrylonitrile graft copolymer, starch-acrylic acid graft copolymer, partially neutralized starch-acrylic acid graft copolymer, saponified vinyl acetate-acrylic ester copolymers, hydrolyzed acrylonitrile or acrylamide copolymers, slightly network crosslinked products of any of the foregoing copolymers, partially neutralized polyacrylic acid, and network crosslinked products of partially neutralized polyacrylic acid. These polymers may be used either independently or in the form of a mixture of two or more monomers, compounds, or the like. Suitable superabsorbents also include the in-situ polymerized superabsorbent as disclosed in the co-pending U.S. patent application Ser. No. 10/017,681, filed 14 Dec. 2001, and U.S. patent application Ser. No. 10/017,760, filed 14 Dec. 2001.

Superabsorbents may be crosslinked partially neutralized polyacrylic acids and starch derivatives therefrom. The superabsorbents may comprise from about 50 to about 90%, particularly about 70% neutralized, network crosslinked, polyacrylic acid. The superabsorbents may be surface crosslinked to provide desired properties needed by the products in use, such properties include gel stiffness, permeability, and absorbency capacity, etc.

The first superabsorbent material may be selected from a wide variety of superabsorbents which meet the saline AUL standard described above. Suitable first superabsorbent polymers include without limitation the alkali metal and ammonium salts of poly(acrylic acid) and poly(methacrylic acid), poly(acrylamides), hydrolyzed maleic anhydride copolymers with vinyl ethers, hydrolyzed maleic anhydride copolymers with alpha-olefins, polyacrylates, polymers and copolymers of vinyl sulfonic acid, and combinations thereof. Further superabsorbent materials include natural and modified natural polymers, such as hydrolyzed acrylonitrile-grafted starch, partially hydrolyzed acrylic acid grafted starch, carboxymethyl cellulose, multicomponent superabsorbent polymers, and combinations thereof.

The second superabsorbent material is selected from superabsorbents that meet the fecal fluid AUL standards set forth above. The second superabsorbent material may also meet the saline AUL standards. The second superabsorbent material can be selected from various materials listed above for the first superabsorbent material, provided that the material meets the fecal fluid AUL standards. Furthermore, the second superabsorbent material in the second zone 64 is different from the first superabsorbent material in the first zone 62. This way, the requirement of the invention is met wherein the first zone 62 of core 40 is designed for urine absorption and the second zone 64 is designed for fecal fluid absorption.

Most superabsorbents do not meet the fecal fluid AUL test. One suitable superabsorbent is available from Stockhausen under the trade name FAVOR 9543. This superabsorbent has a fecal fluid AUL at 0.3 psi of about 20.3 grams/gram and a saline AUL at 0.3 psi of about 25.9 grams/gram. Another is a multicomponent superabsorbent particulate gel from BASF in Ludwigshafen, Germany. One such material, BASF E1231-99, has a fecal fluid AUL of 0.3 psi at 27.1 grams/gram and a saline AUL at 0.3 psi of 33.0 grams/gram. Multicomponent superabsorbent gel particles and methods to prepare them are described in U.S. Pat. Nos. 5,981,689; 6,072,101; 6,087,448; 6,121,409; 6,159,591; 6,194,631; 6,222,091; 6,235,965; 6,342,298; 6,376,072; 6,392,116; 6,509,512; and 6,555,502; U.S. Patent Publications 2001/01312; 2001/07064; 2001/29358; 2001/44612; 2002/07166; 2002/15846; and 2003/14027; and PCT Publications WO 99/25393; WO 99/25745; WO 99/25748; WO 00/56959; WO 00/63295; WO 02/10032; WO 03/18671; and WO 03/37392; the disclosures of which are incorporated by reference.

Other suitable superabsorbents include fibers and particles of crosslinked, partially neutralized polyacrylates, and bicomponent fibers of polyvinyl amine and polyacrylic acid. Suitable superabsorbent particles and fibers can be combined with cellulose pulp fibers, for instance, to form an air-formed or air-laid composite with additional thermal or electromagnetic wave activated binder fibers. The binder fibers may be activated by thermal, electromagnetic, or infrared energy to stabilize the mixture and provide integrity to the absorbent core, or the particular zone of the core. These superabsorbents may also be formed into films or foams.

Table 1 provides fecal fluid AUL values and saline AUL values for several commercial superabsorbent materials determined using the fecal fluid AUL and saline AUL test procedures described herein. Most of the superabsorbents tested passed the saline AUL test at 0.3 psi by absorbing at least about 15 grams/gram of aqueous saline solution, and are thus suitable as the first superabsorbent described above. Only two of the superabsorbents passed the fecal fluid AUL test at 0.3 psi, i.e., absorbed at least about 15 grams/gram of fecal fluid.

TABLE 1
Fecal Fluid And Saline AUL Data For Commercial Superabsorbents

| | | Fecal Fluid AUL | | | | | Saline AUL | | | | |
|---|---|---|---|---|---|---|---|---|---|---|---|
| | Superabsorbent | | 0.01 | 0.3 | 0.6 | 0.9 | | 0.01 | 0.3 | 0.6 | 0.9 |
| Supplier | Type | CRC | psi | psi | psi | psi | CRC | psi | psi | psi | psi |
| Dow Chemical Co. | DRYTECH 2035 | 31.6 | 30.5 | 9.3 | 8.1 | 7.5 | 29.7 | 45.3 | 30.2 | 25.0 | 16.7 |
| Dow Chemical Co. | DRYTECH 535 | 29.5 | 33.8 | 7.3 | 7.4 | 6.6 | 31.8 | 44.8 | 25.8 | 11.5 | 7.7 |
| Dow Chemical Co. | DRYTECH 535 | 33.3 | 26.5 | 7.3 | 5.7 | 5.7 | 31.6 | 44.2 | 22.9 | 9.6 | 7.5 |
| | (212-300 μm) | | | | | | | | | | |
| Dow Chemical Co. | DRYTECH 535 | 28.0 | 31.2 | 10.8 | 8.4 | 8.0 | 29.0 | 42.8 | 24.8 | 10.8 | 8.4 |
| | (600-710 μm) | | | | | | | | | | |
| Stockhausen | FAVOR 880 | 28.8 | 31.2 | 13.1 | 8.4 | 7.0 | 32.8 | 48.7 | 33.8 | 28.4 | 23.2 |
| Stockhausen | FAVOR 9543 | 22.5 | 28.8 | 20.3 | 15.0 | 9.6 | 23.1 | 35.8 | 25.9 | 22.7 | 20.7 |
| BASF | HYSORB P7060 | 30.4 | 31.8 | 9.0 | 7.8 | 6.9 | 32.3 | 48.5 | 32.8 | 27.0 | 18.4 |
| BASF | BASF 1231-99 | 18.3 | 28.2 | 27.1 | 24.1 | 23.3 | 17.1 | 43.9 | 33.0 | 30.0 | 28.6 |
| Sanyo | SANWET KC770 | 29.4 | 34.7 | 9.0 | 7.3 | 6.9 | 31.8 | 46.9 | 31.9 | 26.9 | 22.5 |
| Sanyo | SANWET KC880H | 29.3 | 36.2 | 11.1 | 8.4 | 7.7 | 33.3 | 48.4 | 32.7 | 27.6 | 21.8 |

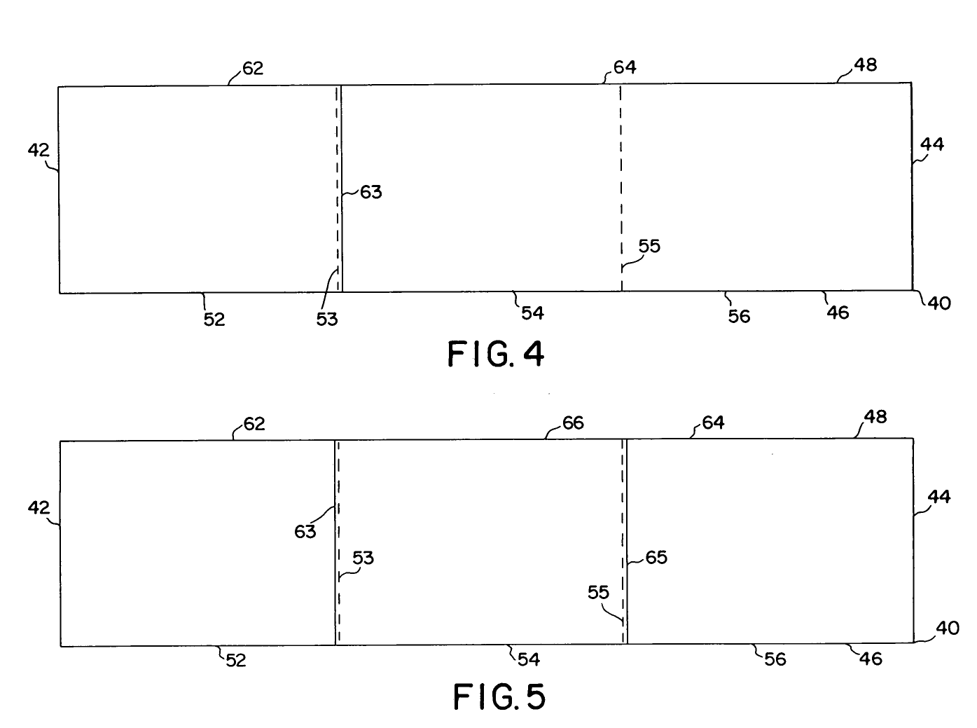

FIGS. 3-5 illustrate alternative embodiments of the heterogeneous absorbent core 40. In the embodiment of FIG. 3, the first zone 62 includes substantially the entire front region 52 and crotch region 54 of the absorbent core 40. The second zone 64 includes only the back region 56 of the core 40. In the embodiment of FIG. 4, the first zone 62 includes only the front region 52 of the absorbent core 40. The second zone 64 includes substantially the entire crotch region 54 and back region 56 of the absorbent core 40.

In the embodiment of FIG. 5, the first zone 62 and second zone 64 are not adjacent to each other. The first zone 62, designed for optimal urine absorption, includes only the front region 52 of the absorbent core 40. The second zone 64, designed for optimal fecal fluid absorption, includes only the back region 56 of the absorbent core 40. The core 40 also includes a third zone 66 including the crotch region 54, and bounded by the first and second zones at boundary lines 63 and 65. The third zone 66 may be designed for optimal absorption of both urine and fecal fluid, and may include superabsorbent materials used in both the first zone 62 and the second zone 64.

The foregoing configurations of heterogeneous absorbent core 40 are merely exemplary. Other configurations, and variations of the configurations illustrated, are also within the scope of the invention. In each case, the first zone 62 and second zone 64 are located in different parts of the absorbent core 40, with the first zone 62 closer to the front waist edge 42 and the second zone 64 closer to the rear waist edge 44.

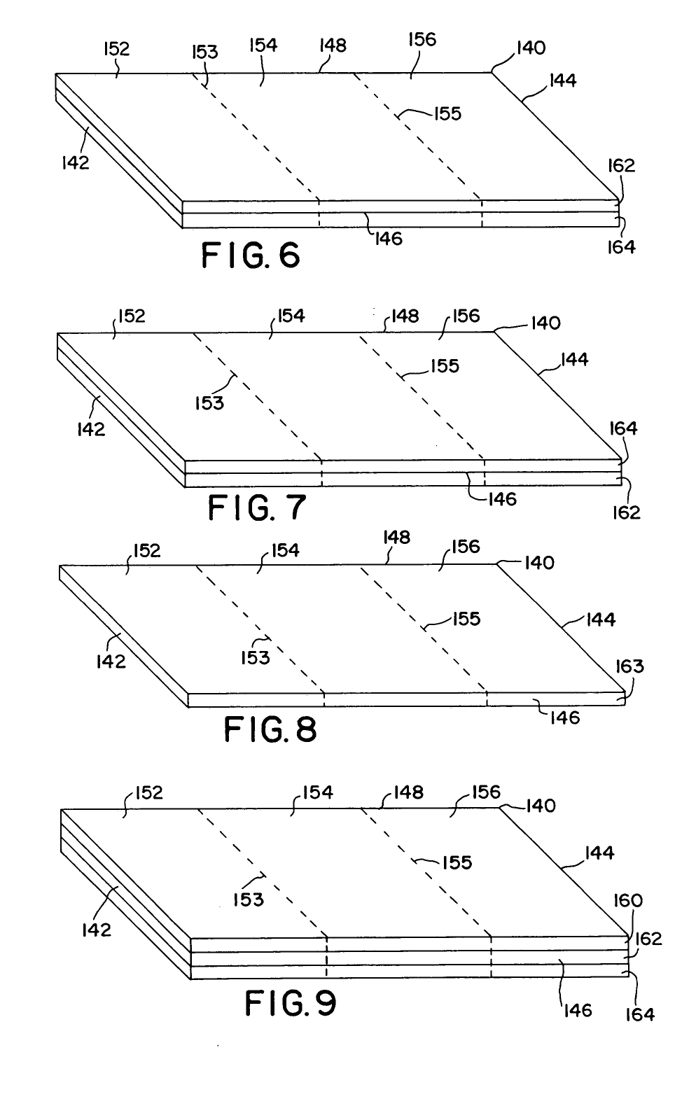

FIGS. 6-9 illustrate different embodiments of a bi-functional absorbent core 140. Referring to FIG. 6, bi-functional absorbent core 140 includes a front region 152, a crotch region 154 and a back region 156 between side edges 146 and 148. The regions 152, 154 and 156 are defined as each including one-third of the total length of the absorbent core 140. The front region 152 commences at the front waist edge 142 of the core 140, and includes the front one-third of the core 140 ending at an imaginary boundary line, shown as a dashed line 153. The back region 156 extends from the rear waist edge 144 of the core 140, and includes the back one-third of the core bounded by the imaginary dashed line 155. The crotch region 154 includes the middle one-third of the length of the core, between the imaginary dashed lines 153 and 155.

The bi-functional absorbent core 140 in FIG. 6 includes a first layer 162 and a second adjacent layer 164. The first layer 162 includes the front region 152 and at least part of the crotch region 154 of the core 140. The second layer 164 includes the back region 156 and at least part of the crotch region 154 of the core 140. The first and second layers 162 and 164 may be coterminous, and may both extend into the front, crotch and back regions.

The first layer 162 includes a first superabsorbent material having a saline AUL of at least about 15 grams aqueous sodium chloride per gram of superabsorbent, particularly at least about 25 grams/gram, particularly at least about 30 grams/gram at a pressure of 0.3 psi, using the saline absorbency under load test described herein. The first superabsorbent material may be dispersed in a cellulose fiber matrix, such as a matrix of wood pulp fluff or fibers, or may be dispersed in another absorbent matrix. The dispersed superabsorbent material may be in the form of particles or fibers. Alternatively, the first superabsorbent material may be in the form of a thin film, foam or other layer. The first superabsorbent material may constitute about 10% to 100% by weight of the first layer 162, particularly about 30% to 100% by weight, particularly about 40% to 100% by weight. When an absorbent fibrous matrix is employed, the first superabsorbent material may constitute about 10% to about 90% by weight of the first layer 162, particularly about 30% to about 80% by weight, more particularly about 40% to about 70% by weight. The absorbent fibrous matrix may constitute some or all of the balance of the weight of the first layer 162. All percentages are based on the dry weights of the ingredients.

The second layer 164 includes a second superabsorbent material, different from the first, which has a fecal fluid AUL of at least about 15 grams fecal fluid per gram of superabsorbent material, particularly at least about 20 grams/gram, particularly at least about 25 grams/gram at a pressure of 0.3 psi, using the fecal fluid absorbency under load test described herein. The second superabsorbent material may also have a saline AUL of at least about 15 grams/gram, particularly at least about 25 grams/gram, particularly at least about 30 grams/gram, using the saline absorbency under load test described herein. The second superabsorbent material may be dispersed in a cellulose fiber matrix, such as a matrix of wood pulp fluff or fibers, or may be dispersed in another absorbent matrix. The dispersed superabsorbent material may be in the form of particles or fibers. Alternatively, the second superabsorbent material may be in the form of a thin film, foam or other layer. The second superabsorbent material may constitute about 10% to 100% by weight of the second layer 164, particularly about 30% to 100% by weight, particularly about 40% to 100% by weight. When an absorbent fibrous matrix is employed, the second superabsorbent material may constitute about 10% to about 90% by weight of the second layer 164, particularly about 30% to about 80% by weight, more particularly about 40% to about 70% by weight. The absorbent fibrous matrix may constitute some or all of the balance of the weight of the second layer 164. Again, all percentages are based on the dry weights of the ingredients.

As explained above, the first superabsorbent material in the first layer 162 and the second superabsorbent material in the second layer 164 may be combined with other superabsorbent materials and other ingredients, provided that the indicated percentages of the first superabsorbent material in the first layer and the second superabsorbent material in the second layer are satisfied. Also, the type and amount of the additional materials should not prevent the superabsorbent materials from receiving or absorbing the liquids as intended.

FIGS. 7-9 illustrate alternative embodiments of the bi-functional absorbent core 140. In the embodiment of FIG. 7, the order of layers 162 and 164 are reversed from the embodiment of FIG. 6. Thus, the second layer 164 including the second superabsorbent material designed for absorption of fecal fluid acts as the first liquid receiving layer, while the first layer 162 including the first superabsorbent material designed for urine absorption acts as the second liquid receiving layer. In the embodiment of FIG. 7, the layers 162 and 164 may have any of the same alternative constructions described above for the layers 162 and 164 in the embodiment of FIG. 6. The only difference is that the order of the layers is reversed.

In the embodiment of FIGS. 6 and 7, the first layer 162 including the first superabsorbent material and the second layer including the second superabsorbent material are superimposed along the entire length of bi-functional absorbent core 140. By having the layers containing the first and second superabsorbent materials present along the entire length, all three regions 152, 154 and 156 of the absorbent core exhibit bi-functional optimal absorption of both urine and fecal fluids. Other embodiments are also contemplated by the invention. For purposes of the invention, at least the crotch region 154 should exhibit bi-functional absorption along its length, and should contain both types of superabsorbent along its length. In one embodiment, the first superabsorbent material may be present in only the front region 152 and the crotch region 154, and the second superabsorbent material may be present in only the crotch region 154 and the back region 156. In another embodiment, the first superabsorbent material may be present in the front region 152, crotch region 154 and back region 156, and the second superabsorbent material may be present in only the crotch region 154 and the back region 156. In another embodiment, the first superabsorbent material may be present in only the front region 152 and the crotch region 154, and the second superabsorbent material may be present in the front region 152, crotch region 154 and back region 156.

FIG. 8 illustrates another embodiment of the bi-functional core 140 in which the first and second superabsorbent materials are both combined in a single layer 163. In this embodiment, each of the first and second superabsorbents should constitute at least about 10% by weight of the layer 163, particularly at least about 15% by weight, particularly at least about 20% by weight. The combined weight of the first and second superabsorbent materials may be as high as 100% by weight of the layer 163, or may range from about 20% to about 90% by weight, or from about 30% to about 80% by weight, or from about 40% to about 70% by weight when an absorbent cellulose fiber matrix is present. The superabsorbent materials may also be in the form of a film, foam, bi-component fiber web, or any other form previously described.

FIG. 9 illustrates an alternative embodiment of a bi-functional core 140 which includes three layers. In the embodiment of FIG. 9, neither the first layer 162 including the first superabsorbent polymer, nor the second layer 164 including the second superabsorbent polymer, serves as the first liquid receiving layer. Instead, layer 160 serves as the first liquid receiving layer. Layer 160 may be formed of an absorbent fibrous web, such as cellulose fluff or pulp, and may be devoid of superabsorbent material. Layer 160 may serve to receive a liquid insult, and distribute it horizontally before the liquid reaches the superabsorbent-containing storage layers 162 and 164.

The foregoing configurations of bi-functional absorbent core 140 are merely exemplary. Other configurations, and variations of the configurations illustrated, are also within the scope of the invention. In each case, the first and second superabsorbent materials should both be present at least along the length of the crotch region 154 of the absorbent core 140, so that at least the crotch region is designed for both urine absorption and fecal fluid absorption.

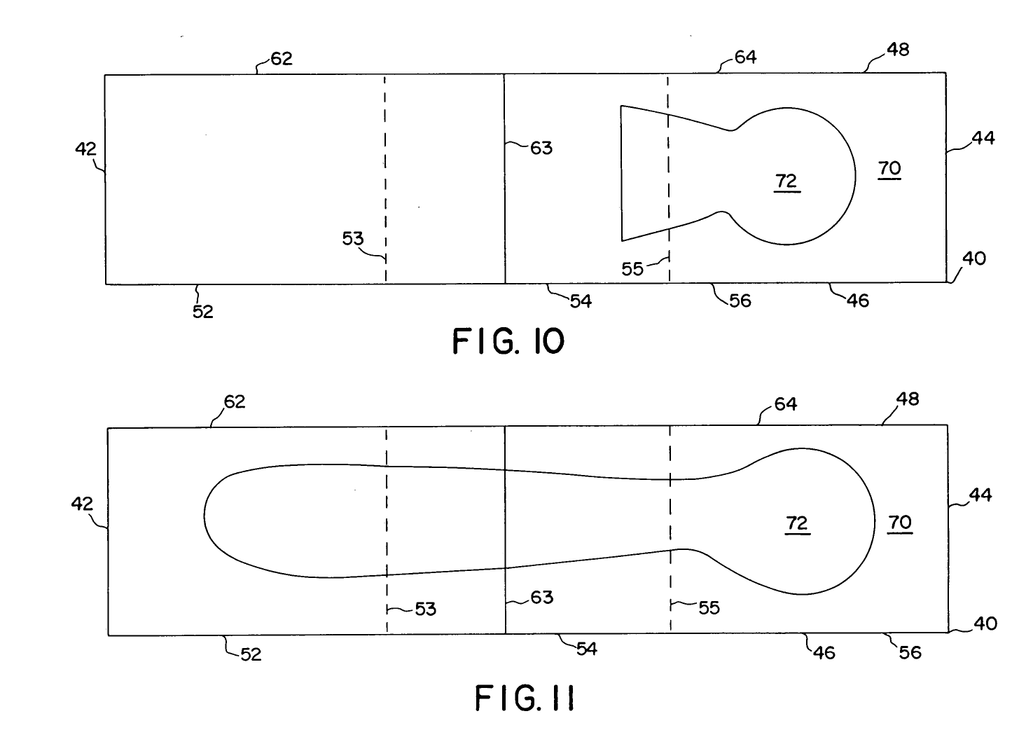

FIGS. 10 and 11 illustrate absorbent cores 40 which correspond to the absorbent core 40 of FIG. 2, with the same reference numerals indicating the same structural elements, with the following improvement. In the absorbent core 40 of FIG. 10, the back region 56 and at least part of the crotch region 54 (i.e., the second zone 64) include a relatively raised edge region 70 and a relatively depressed region 72. The depressed region 72 may have the shape of a keyhole, oval, rectangle, circle, square, or other suitable shape. The absorbent core 40 of FIG. 11 also includes a relatively raised edge region 70 and a relatively depressed region 72. In this embodiment, the raised and depressed regions extend from the back region 56 to the front region 52 of the absorbent core (i.e., in both zones 62 and 64). The depressed region 72 in FIG. 11 may also have a variety of shapes.

The relatively depressed region 72 surrounded by the relatively raised region 70 provides a physical mechanism to receive and contain fecal fluid, and prevent it from escaping along the edges of the absorbent core 40. A suitable material to intake and entrap BM may be provided in the depressed region. The depressed region 72 may be provided with honeycomb structures, nets, scrims, apertured materials or other grid-like structures, open nonwoven webs, or materials with a topographical structure which further restrict the lateral flow of fluids. When the depressed region 72 extends into the front region 52 of the absorbent core as shown in FIG. 11, it may contain and restrict the flow of urine as well as fecal fluid.

The heterogeneous absorbent core 40 of any of FIGS. 2-5 and 10-11 may be formed using a matrix material which has a more open structure in the regions used for receiving the fecal fluid (liquid) component of runny BM. For instance, the absorbent matrix material may be formed from a more open structure in the back region 56, the crotch region 54, or both, of the absorbent core 40. Open matrix materials include lofty nonwoven webs such as through air bonded carded webs, lofty through air bonded bicomponent spunbond webs, and other rapid intake materials for high viscosity liquids. The lofty nonwoven webs typically have densities of about 0.1 grams per cubic centimeter or less, particularly about 0.06 g/cc or less. One suitable through air bonded carded web has a basis weight of about 75 grams per square meter and a density of about 0.024 g/cc. Other open matrix materials include materials with raised surface textures such as flocked materials, materials with hairy surfaces, corrugated nonwoven materials, honeycomb materials, and materials such as films or nonwoven webs with apertures or open holes. One suitable open material is a honeycomb material available from VersaCore Industrial Corporation. Apertured materials may be layered and bonded together by an adhesive or other means, to form an open structure. Other open matrix materials include open celled foams, reticulated foams, and structures cut into irregular shapes that are joined together to form an open structure.

The open matrix material can be combined with the second superabsorbent material, or a mixture of the first and second superabsorbent materials. The superabsorbent material(s) can be entrapped in the open matrix material, or attached using an adhesive or a latex binder material to the interior, surface(s) or both of the open matrix material. The open matrix material can be coated with the superabsorbent material(s).

The open matrix material should have a thickness sufficient to separate the fecal fluid from the skin, in the range of about 0.5 mm to about 20 mm, particularly about 1 to about 15 mm, particularly about 2 to about 10 mm. The open matrix material should have pores, void spaces or other openings at least 100 microns in equivalent diameter (which is the diameter of a circle with the same area), suitably at least 200 microns in equivalent diameter. The open matrix material can have an open volume of at least 20%, particularly at least 40%, suitably at least 50%. These properties should be present in the material during or shortly after BM insult occurs, but need not be present originally. The openings or void spaces may be present during manufacture of the absorbent core, or may be triggered during use by mechanical means, temperature, motion or liquid. The openings or voids may be of uniform or nonuniform size and distribution, and may provide a continuous or tortuous path through the matrix material. The remainder of the absorbent core 40 may be formed of another, less open matrix material designed for lower viscosity aqueous liquid such as urine.

The bifunctional absorbent core 140 of any of FIGS. 6-9 may also be formed using a more open absorbent matrix material in desired regions. For instance, the first (upper) layer 162 in FIG. 6, the second (upper) layer 164 in FIG. 7, or the layer 160 in FIG. 9 may be formed using a more open absorbent matrix material than the lower layer(s) in the absorbent core. Furthermore, any of the absorbent cores of FIGS. 2-11 may include an upper layer in the crotch region and/or back region which is more open than a lower layer in the same region(s).

Referring again to FIG. 1, the other components of the personal care article 25, such as a disposable absorbent article, may be of conventional structures. Surge layer 43 and body-side liner 50 are constructed from highly liquid pervious (generally non-absorbent) materials. These layers function to transfer liquid from the wearer to the interior of the absorbent article. Suitable liquid pervious materials include porous woven materials, porous nonwoven materials, films with apertures, open-celled foams, and batting. Examples include, without limitation, any flexible porous sheets of polyolefin fibers, such as polypropylene, polyethylene or polyester fibers; webs of spunbonded polypropylene, polyethylene or polyester fibers; webs of rayon fibers; bonded carded webs of synthetic or natural fibers or combinations thereof. U.S. Pat. No. 5,904,675, issued 18 May 1999 to Laux et al., and incorporated by reference, provides further examples of suitable surge materials. Either layer may also be an apertured plastic film.

In order for the absorbent core 40 to be most effective, the upper layers of the absorbent structure (e.g., the bodyside liner 50 and surge layer 43 in FIG. 1) should each have a structure which is relatively open and receptive to the transmission of viscous fluids. Open structures include, for instance, apertured materials, open low density spunbond materials, and other open nonwoven materials.

Open structures for the upper layers provide better intake of runny BM and facilitate travel of the runny BM to a location away from the wearer's skin. These structures also help prevent leakage from the absorbent article. In one embodiment, the bodyside liner and/or surge material layers may have open structures only in those regions which superimpose the region(s) of the absorbent core designed for optimal fecal fluid absorption. For instance, the bodyside liner and/or surge layers may have open structures in regions which superimpose the back region 56, the crotch region 54, or both of the absorbent core 40. The remainder of the bodyside liner and/or surge layers may have a more conventional structure designed primarily for the intake of less viscous aqueous liquid, such as urine.

Outer cover 30 may be liquid impermeable, and is suitably breathable to water vapor. Outer cover 30 may include a breathable, substantially liquid impermeable polymer film formed by mixing a thermoplastic polyolefin with a particulate inorganic filler, and stretching the film in at least one direction to form voids around the filler particles and micropores in the film. Outer cover 30 may also include a fibrous nonwoven web, such as a polyolefin spunbond web laminated to the breathable film, to provide a soft, cloth-like texture and feel to the underside of personal care article 25.

Attached to outer cover 30 are waist elastics 26, leg elastics 31, and fastening tabs 28, which may be of any conventional structure. Leg elastics 31 may include a carrier sheet 32 and individual elastic strands 34. Fastening tabs 28 may include fastening tapes or mechanical fasteners such as VELCRO hook and loop fastening elements.

### SALINE AUL TEST

The saline absorbency under load test is designed to estimate how much urine is absorbed by a material. The saline AUL test uses an aqueous solution containing 0.9% by weight sodium chloride.

Equipment and Materials:

a) Electronic balance, accurate to 0.01 grams;

b) Cylinder: 1 inch (25.4 mm) inside diameter plastic cylinder with 400 mesh stainless steel screen fused into the cylinder bottom; 4.4 gram plastic piston at 0.995 inch diameter (0.005 inch less than the cylinder's inside diameter);

c) 0.9% saline;

d) Fluid basin with a 3×3 in² area per one cylinder group;

e) Timer that can read up to sixty minutes by seconds;

f) SCOTT® brand paper toweling used for blotting;

g) Weights (100.29 grams, 200.57 grams and 300.85 grams).

Procedure:

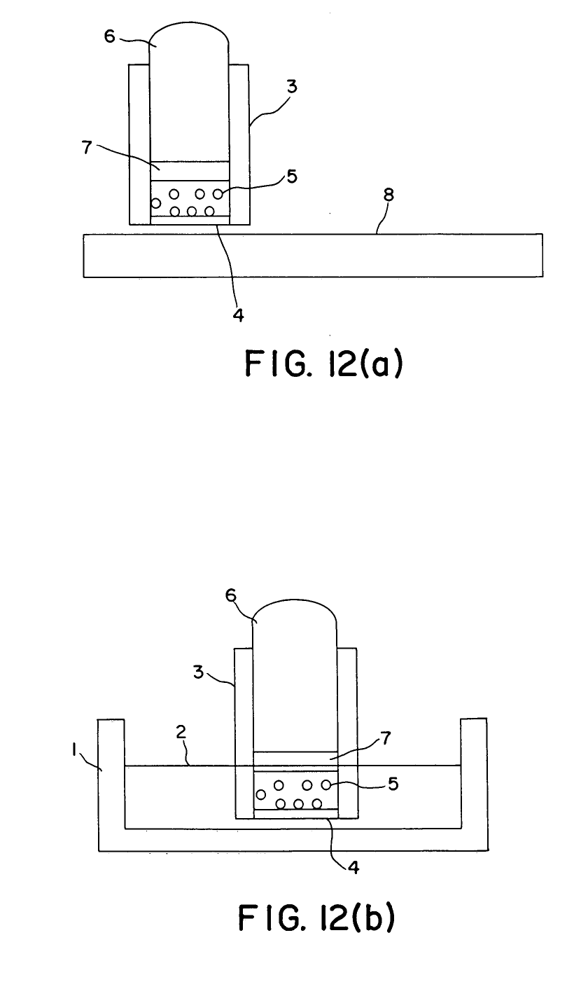

Referring to FIG. 12(a), weigh out 0.160 g superabsorbent 5 within 0.001 g directly into the plastic cylinder 3 with the 400-mesh screen 4 using balance 8, and install cap 7. Be careful not to contact the superabsorbent with the sides of the cylinder because the granules may adhere to the sides. Gently tap the cylinder 3 until the superabsorbent granules are evenly distributed on the 400-mesh screen 4.

Place the plastic piston 6 in the cylinder and place any weight over the plastic piston (no weight for 0.01 psi, 100.29 g for 0.3 psi, 200.57 g for 0.6 psi, 300.85 g for 0.9 psi). Weigh the device with the weight and the superabsorbent and record as the total weight of the system.

Set up devices to run 2 repetitions of samples with each pressure amount (0.01, 0.3, 0.6 and 0.9 psi).

Referring to FIG. 12(b), place each cylinder in a fluid basin 1 with 20 ml of 0.9% saline solution 2. After 3 minutes remove the device and blot on SCOTT® brand paper toweling three times in different areas for 1 second each. Weigh the cylinder and record the weight. Return cylinder device to its fluid basin. Keep a timer running throughout the test (weighing takes about 10 seconds).

Take readings at 3, 5, 10, 15, 30, 45, and 60 minutes. Use a fresh SCOTT® brand paper towel blotted three times in different areas for one second, for each reading.

Calculate the grams of fluid absorbed per gram of superabsorbent and plot as a function of elapsed time (this includes blot and weigh time). The reported saline AUL at each pressure is the maximum amount of saline absorbed per gram of superabsorbent at 60 minutes.

### PREPARATION OF SYNTHETIC FECAL FLUID

In order to develop a successful fecal fluid simulant, the resultant fecal fluid simulant should have key properties similar to those of the real fecal fluid. But the real biological fecal fluids have huge inherent variations. The feces of infants vary substantially depending on the type of food and among infants. The infants on formula produce feces of much higher viscosity than the infants on mother's breast milk. To obtain the BM properties of runny BM, a number of infants on breast milk were recruited. Their feces were colleted with a special diaper with a BM collection bag. The collected samples were tested for their viscosities and other properties.

A. Determination of the fecal fluid property targets:

1. Separation of Infant BM

In order to determine the target for fecal fluid simulant, it was important to separate the fecal fluid (which is the liquid portion of the BM) from the collected BM samples and then the properties of fecal fluid can be determined. To accomplish this, a centrifuge separation method was used. This method worked well. It resulted in two fractions, a solid fraction and a fecal fluid fraction. The fecal fluid fraction was collected and subjected to the analysis of chemical compositions and testing of its interaction with superabsorbent. A total of nineteen fecal fluid samples were collected in a six-week period.

2. Composition of Fecal Fluid

Nineteen collected fecal fluid samples were frozen and analyzed for composition. Samples of several BM samples were also analyzed for internal control prior to separation of fecal fluid. The following results were found:

Protein:

Average, 1.99%; Standard Deviation: 0.44%; Range, 1.48 to 2.83%

Carbohydrates:

Average, 6.84%; Standard Deviation, 2.11%; Range, 4.7 to 11.3%

Fat:

Average, 0.11%; Standard Deviation, 0.21%; Range, 0.01 to 11.3%

Water:

Average, 90.82%; Standard Deviation, 2.3%; Range, 85.84 to 93.48%

The compositional data were used to determine the effects of these fecal fluid components on the absorbency of superabsorbents and develop a fecal fluid simulant.

3. Absorbency of Collected Fecal Fluid

The absorbency of fecal fluid was determined using the fecal fluid absorbency under load (AUL) method at 0.3 psi, described below. The fecal fluid samples did not contain any particles but have dissolved proteins, carbohydrates, and a very small amount of fat. The viscosity values of the collected fecal fluids were under 1 poise.

The screen porosity of the AUL calendar was found to be important to obtain reproducible results. The 100-mesh screen was found to be effective. A 400-mesh screen was found to be too fine for obtaining reproducible results partly caused by the increased resistance to the transport of fecal fluid through the small pores on the screen.

Fourteen collected fecal fluid samples were tested for 0.3 psi AUL. A Stockhausen superabsorbent (FAVOR 880) was used in the test. The average value of AUL for all the samples was 9.6 g/g (the viscosity of all the BM samples range from 1.4 to 109.9 poise).

The fecal fluid samples were also grouped according to the viscosity of whole BM prior to separation. The fecal fluid separated from the low viscosity (20 poise or less) BM had an average 0.3 psi AUL value of 13.4 g/g for FAVOR 880 while fecal fluid separated from the medium to high viscosity (20 to 109.9 poise) BM had an average of 0.3 psi AUL of 6.7 g/g. Therefore, there is a correlation between the fecal fluid AUL value and the original viscosity of whole BM. This is probably caused by the difference in the soluble material content in the samples. The high viscosity samples had a high level of dissolved proteins, carbohydrates, etc. These dissolved components also contribute to the depression of AUL by fecal fluid. This was illustrated by the component effect data disclosed in the next section.

With these determined targets, it was possible to proceed to the next step in the invention of a fecal fluid simulant.

B. Determination of the Effect of Fecal Fluid Components on the Absorbency

In order to develop a fecal fluid simulant, it was important to determine the quantitative effect of the individual component on the absorbency.

1. Effect of Protein

The proteins from both natural and synthetic origins can be used. An example of natural protein is egg white. Egg white can be separated into two fractions: a thin egg white fraction of low molecular weight and low viscosity, and thick egg white fraction of high viscosity and containing mucin.

Synthetic proteins prepared by polymerization of a variety of amino acids using protein synthesizer (employing Meerifiled's polypeptide synthesis process) can be utilized. The synthetic proteins have precise chemical composition and amino acid sequence but they are costly to make and less available.

For this invention, various egg components were separated and used as model compounds for protein. The egg components had the advantages of being biologically produced, low cost and safe to use.

The 0.3 psi fecal fluid AUL of pure egg components were determined to be as follows:

Thin egg white: 4.3 g/g

Thick egg white: 3.2 g/g

Egg yolk: 4.1 g/g

To determine the effect of egg protein on AUL, a series of solutions containing proteins were made. These solutions had egg protein concentrations in the range of protein content in the collected infant fecal fluids. Three concentration levels were selected: 1.4% (representing the low end of protein content of collected fecal fluids); 2.3% (representing the average of the protein content of collected fecal fluids), and 3.0% (representing the high end of the protein concentration of collected fecal fluids).

The solutions were based on 0.9% saline. Since egg whites contain water, an egg protein solution of certain protein concentration and salt concentration was needed.

The proper concentration was determined by first determining the water content of egg component using a moisture analyzer. The water content was then translated into the protein content in each egg component. The water in the egg component was taken into consideration when egg protein was added to the solution. The water in egg will cause a dilution in sodium chloride content. Additional sodium chloride was added to the solution based on the compositional calculation to obtain a composition of base ingredients going into the solution.

The effect of thin egg white protein on the absorbency of FAVOR 880 was determined. Thin egg white contains low molecular weight protein. It does not contain the high viscosity mucin. The FAUZL (free absorbency under zero load) decreased slightly with the increasing thin egg white protein. The fecal fluid AUL at 0.3 psi decreased substantially with increasing egg white protein, from 28.5 g/g at 0% thin egg white in 0.9% aqueous saline solution to 13.6 g/g at 3.0% egg white in aqueous saline solution.

The effect of thick egg white protein on both the FAUZL and AUL was determined. Thick egg white contains the high viscosity mucin component. The thick egg white decreased the fecal fluid AUL values more severely than the thin egg white at the same protein concentration. The fecal fluid AUL at 0.3 psi decreased with increasing egg white protein, from 28.5 g/g, 20.8 g/g, 16.9 g/g and 13.6 g/g, respectively, at 0%, 1.4%, 2.2% and 3.0% thin egg white in aqueous saline solution, respectively. The relationship was used in developing the fecal fluid simulant.

2. Effect of Carbohydrates on the Absorbency

The effect of carbohydrates on fecal fluid AUL and FAUZL was determined by making testing fluid containing model carbohydrates. All the experiments were performed in 0.9% saline. There was little effect on absorbency resulting from carbohydrates.

The effect of sucrose (formed from two glucose units) on fecal fluid AUL and FAUZL was determined. The effect of this carbohydrate on both FAUZL and fecal fluid AUL was minimal. The effect of corn syrup on absorbency was determined. The effect was also negligible on both fecal fluid AUL and FAUZL.

Among the carbohydrates studied, the only carbohydrate having a substantial effect on the absorbency was dextran. Dextran is a bacterially produced polysaccharide from sucrose. It has different molecular weights depending on the bacteria strains and conditions used for making dextrans. It was found that the FAUZL was reduced from 36.2 g/g for FAVOR 880 in 0.9% aqueous saline with no dextran to 25.8 g/g in 0.9% aqueous saline at 12% dextran concentration (the high end of determined carbohydrates in fecal fluid). The saline AUL was decreased from 28.5 g/g for 0.9% aqueous saline and no dextran to 19.1 g/g for 0.9% aqueous saline and 12% dextran.

3. Effect of Fat on Absorbency

When emulsified corn oil (used as a fat simulant) was added to the saline solution, it was found that the fat had little effect on either fecal fluid values.

C. Fecal Fluid Simulant Formulations

Based on the above relationship between the fecal fluid component and the determined absorbency, a series of formulation experiments were performed to develop a viable fecal fluid simulant with properties similar to the “real” biologically produced fecal fluid.

The fecal fluid AUL of saline, low viscosity fecal fluid, medium to high viscosity fecal fluid, and various egg components were determined. The real fecal fluid had AUL values between those of 0.9% saline and the egg components.

A series of formulations were designed based on calculation of the fecal fluid component effect at different concentrations. It was found that both natural and synthetic carbohydrates can be used. Low molecular weight carbohydrates, carbohydrate oligomers, and high molecular weight carbohydrates can be used in the formulation of the fecal fluid simulant.

4. Embodiments of Fecal Fluid Simulants

The fecal fluid simulants comprise proteins, carbohydrates, salt and water. Proteins from various origins and different preparation methods can be used for this invention. Proteins separated from eggs such as thin egg white, thick egg white, egg yolk, mixtures of egg white and yolk, and plasma separated from human blood or animal blood can be used as the protein component in the fecal fluid simulants. The range of protein ranges from 0.1 percent to 10% by weight of the simulant.

Various carbohydrates can also be used in the formulations. The amount of carbohydrates range from 0.1 to 15% by weight. The preferred carbohydrate is dextran.

Salts of monovalent, divalent and multi-valent metal ions and inorganic anions can be used in this invention. Examples of metal ions are sodium, potassium, lithium, magnesium, calcium ions, etc. Examples of inorganic anions are chloride, bromide, fluorides, sulfate, sulfonate, phosphate, carbonate, etc. The amount of the salt level can be adjusted to the average level of salt found in the fecal fluids.

The fecal fluid simulant formulation can be based on both saline or distilled water. In the case of distilled water, additional salts are used to adjust the ionic strength of real fecal fluid.

The resulting fecal fluid is homogeneous without any observable phase separation. The resulting fecal fluid is typically has a light yellow color.

The stability of the fecal fluid simulant can be substantially increased by adding preservatives.

### EXAMPLE 1

In a 1 liter PYREX glass beaker, 128.5 grams of 0.90% (w/w/) aqueous solution of sodium chloride supplied by RICCA® Chemical Company, Arlington, Tex., 10 L bag) was added. A magnetic stirrer was placed in the beaker and set on a magnetic stirring plate (Nuova II Stir Plate, Thermolyne Corporation, a subsidiary of Sybron Corporation, Dubuque, Iowa) on medium high speed (Level 7), 0.45 grams of sodium chloride (supplied by Aldrich Chemical Company, Milwaukee, Wis.) was added to the same beaker. After the sodium chloride completely dissolved, 0.72 grams of dextran (supplied by SIGMA® Chemical Company, St. Louis, Mo.) was subsequently added to the solution. After the dextran completely dissolved, 50 grams of thin egg white was added to the solution (separated from eggs by first removing the egg yolk and then filtering the egg through a 1700-micron filter made by American Scientific Products, McGaw Park, Ill.). Once all the thin egg white was added, the solution was mixed for 20 minutes. At the end of the mixing process, the beaker was removed from the magnetic stirring plate. Some of the egg particles coagulated to form pliable, stringy or clumpy, solid white masses on the center surface of the solution. The masses were removed using metal tweezers. The process produced a visually homogeneous liquid that is a pale, golden-yellowish in color.

AUL testing was performed by placing approximately 0.160 grams of a superabsorbent FAVOR 880 from Stockhausen in an AUL cylinder with a 100-mesh screen under a pressure of 0.3 psi. The cylinder was then set directly into the test fluid. Weight gains of the superabsorbent at different times were measured by removing the cylinder from the fluid and blotting away the excess fluid with a towel.

The following fecal fluid AUL result was obtained based on the average values of two repetitions using the simulant made in this example (Low Viscosity Average 1:LVA1): Absorbency under load at 0.3 psi: 13.1 g/g

The targeted average absorbency for real, low viscosity fecal fluid: Absorbency under load at 0.3 psi: 13.4 g/g/ (range: 11.2-17.2 g/g).

### EXAMPLE 2

The same 1 liter PYREX glass beaker as in Example 1 was used. In the beaker, 173.7 grams of 0.90% (w/w) aqueous solution of sodium chloride was added, followed by 0.52 grams of sodium chloride and 57.8 grams of thick egg white. The same mixing process as Example 1 was used. The process produced a visually homogeneous liquid that is a pale, golden-yellowish color.

The following properties were obtained based on the average values of two repetitions using the simulant made in this example (All Samples Average 3:ASA3): Absorbency under load at 0.3 psi: 10.0 g/g. The targeted average real fecal fluid absorbency for all viscosity levels: Absorbency under load at 0.3 psi: 9.6 g/g (range: 5.8-17.2 g/g).

### EXAMPLE 3

The same 1 liter PYREX glass beaker as in Example 1 was used. In the beaker, 123.9 grams of 0.90% (w/w) aqueous solution of sodium chloride was added, followed by 0.63 grams of sodium chloride and 70 grams of thick egg white. The same mixing process as Example 1 was used. The process produced a visually homogeneous liquid that is a pale, golden-yellowish color.

The following AUL result was obtained based on the average values of two repetitions using the fecal fluid simulant made in this example (High Viscosity Average 2: HVA2): Absorbency under load at 0.3 psi: 6.5 g/g. The targeted average absorbency for real, high viscosity fecal fluid: Absorbency under load at 0.3 psi: 6.7 g/g (range: 5.8-8.4 gig).

### EXAMPLE 4

The same 1 liter PYREX glass beaker as in Example 1 was used. In the beaker, 200 grams of 0.90% (w/w) aqueous solution of sodium chloride was added, followed by 0.45 grams of sodium chloride and 50 grams of thick egg white. The same mixing process as Example 1 was used. The process produced a visually homogeneous liquid that is a pale, golden-yellowish color.

The following AUL result was obtained based on the average values of two repetitions using the fecal fluid simulant made in this example (Low Viscosity Low 2: LVL2): Absorbency under load at 0.3 psi: 10.9 g/g. The targeted low end of absorbency for real, low viscosity fecal fluid: Absorbency under load at 0.3 psi: 11.2 g/g (range: 11.2-17.2 g/g).

### EXAMPLE 5

The same 1 liter PYREX glass beaker as in Example 1 was used. In the beaker, 160 grams of 0.90% (w/w) aqueous solution of sodium chloride was added, followed by 0.38 grams of sodium chloride and 40 grams of thin egg white. The same mixing process as Example 1 was used. The process produced a visually homogeneous liquid that is a pale, golden-yellowish color.

The following AUL result was obtained based on the average values of two repetitions using the fecal fluid simulant made in this example (Low Viscosity High 2: LVH2): Absorbency under load at 0.3 psi: 16.6 g/g. The targeted high end of absorbency for real, low viscosity fecal fluid: Absorbency under load at 0.3 psi: 17.2 g/g (range: 11.2-17.2 g/g).

### FECAL FLUID AUL TEST

To measure the absorbency under load for fecal fluid, a fecal fluid simulant referred to as “LVA1” can be used. The procedure for making LVA1 can be as follows:

In a 1 liter PYREX glass beaker, 128.5 grams of 0.90% (w/w) aqueous solution of sodium chloride supplied by (RICCA® Chemical Company, Arlington, Tex., 10 L bag) is added. A magnetic stirrer is placed in the beaker and set on a magnetic stirring plate (Nuova II Stir Plate, Thermolyne Corporation, a subsidiary of Sybron Corporation, Dubuque, Iowa) on medium-high speed (Level 7). Then, 0.45 grams of sodium chloride (supplied by Aldrich Chemical Company, Milwaukee, Wis.) is added to the same beaker. After the sodium chloride completely dissolves, 0.72 grams of dextran (supplied by SIGMA® Chemical Company, St. Louis, Mo.) is subsequently added to the solution. After the dextran completely dissolves, 50 grams of thin egg white is added to the solution (separated from eggs by first removing the egg yolk and then filtering the egg through a 1700-micron filter made by American Scientific Products, McGaw Park, Ill.). Once all the thin egg white is added, mix the solution for 20 minutes. At the end of the mixing process, remove the beaker from the magnetic stirring plate. Many times, some of the egg particles coagulate and form pliable, stringy or clumpy, solid white masses on the center surface of the solution. Remove the masses using a metal tweezers. The process should produce a visually homogeneous liquid that is a pale, golden-yellow in color.

AUL testing for the simulated fecal fluid and real fecal fluid was performed by placing approximately 0.160 grams of a superabsorbent, FAVOR 880 from Stockhausen, in an AUL cylinder with a 100-mesh screen under a pressure of 0.3 psi. The cylinder was then set directly into the test fluid. Weight gains of the superabsorbent at different times were measured by removing the cylinder from the fluid and blotting away the excess fluid with a towel.

The following AUL results were obtained based on the average values of two repetitions using the simulant made in this example (Low Viscosity Average 1:LVA1):

Absorbency under load of the fecal fluid simulant at 0.3 psi: 13.1 g/g

Absorbency under load of real, low viscosity fecal fluid at 0.3 psi:

13.4 g/g (average)

Based on the foregoing, it was determined that the fecal fluid simulant is similar to actual fecal fluid for purposes of AUL testing. The fecal fluid AUL for various superabsorbents was bested using an apparatus similar to the one described above for saline AUL testing, incorporating the following specific equipment and procedural steps.

Equipment and Materials:

a) Electronic balance, accurate to 0.01 grams;

b) Cylinder: 1 inch (25.4 mm) inside diameter plastic cylinder with 100 mesh stainless steel screen fused into the cylinder bottom; 4.4 gram plastic piston at 0.995 inch diameter (0.005 inch less than the cylinder's inside diameter);

c) LVA1 Simulant;

d) Fluid basin with a 3×3 in area per one cylinder group;

e) Timer that can read up to sixty minutes by seconds;

f) SCOTT® brand paper toweling used for blotting;

g) Weights (100.29 grams, 200.57 grams and 300.85 grams).

Procedure:

Referring to FIG. 12(a), weigh out 0.160 g superabsorbent 5 within 0.001 g directly into the plastic cylinder 3 with the 100-mesh screen 4 using balance 8, and install cap 7. Be careful not to contact the superabsorbent with the sides of the cylinder because the granules may adhere to the sides. Gently tap the cylinder 3 until the granules are evenly distributed on the 100-mesh screen 4.

Place the plastic piston 6 in the cylinder and place any weight over the plastic piston (no weight for 0.01 psi, 100.29 g, for 0.3 psi, 200.57 g for 0.6 psi, 300.85 g for 0.9 psi). Weigh the device with the weight and the superabsorbent and record as the total weight of the system.

Set up devices to run 2 repetitions of samples with each pressure amount (0.01, 0.3, 0.6 and 0.9 psi).

Referring to FIG. 12(b), place each cylinder in a fluid basin 1 with 20 ml of fecal fluid simulant LVA1 (reference numeral 2). After 3 minutes remove the device and blot on SCOTT® brand paper toweling three times for 1 second each. Weigh the cylinder and record the weight. Return cylinder device to its fluid basin. Keep a timer running throughout the test (weighing takes about 10 seconds).

Take readings at 3, 5, 10, 15, 30, 45, and 60 minutes. Use a fresh SCOTT® brand paper towel for each reading.

Calculate the grams of fluid absorbed per gram of superabsorbent and plot as a function of elapsed time (this includes blot and weigh time). The fecal AUL is the maximum amount of fecal fluid absorbed per gram of superabsorbent.

### CRC SCREEN TEST

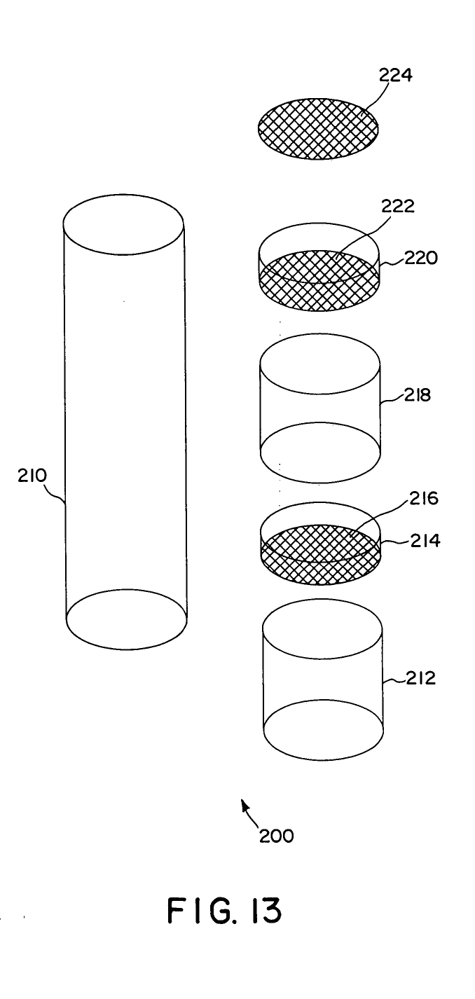

The CRC screen test is a way to evaluate the retention of liquid (e.g., fecal fluid) in a saturated superabsorbent polymer when the saturated superabsorbent polymer is exposed to pressure exerted by a centrifuge. An apparatus used for this test is illustrated (in exploded view) in FIG. 13. Referring to FIG. 13, apparatus 200 includes a cylindrical centrifuge 210 having a hollow interior with a diameter of about 50 min. Stacked inside the cylinder 210 are a lower spacer 212, an optional ring 214 including a mesh screen 216 having 112-μm openings, an upper spacer 218, an upper ring 220 including a mesh screen 222 having 160-μm openings, and a mesh cover screen 224 having 210 micron openings. The superabsorbent being evaluated is placed in the ring 220, between the screens 222 and 224. The upper ring 220 and screen 222 are collectively referred to as the “holder,” and screen 224 is referred to as the “cover.”

To perform the CRC test, weigh the ring and cover screen together. This is the weight for the “holder and screen” section of the data sheet. Next, remove the cover screen and weigh only the holder. Take out the weight of the holder. Put 0.04 grams (40 mg) of the superabsorbent into the holder to measure the separated particle weight of the superabsorbent. Record the weights on the data sheet. Spread particles out as evenly as possible. Perform four (4) replicates each of the 40-mg test for each superabsorbent tested.

Put 20 ml of a fecal fluid simulant (e.g., LVA1) in the specific cup designed for the test to do the 40-mg test. Make sure the fecal fluid simulant completely covers the top screen so superabsorbent can absorb fluid from top and bottom. Cover the top of the dish so evaporation cannot occur. Allow the superabsorbent to soak in the fecal fluid simulant for 30 minutes.

After the superabsorbent has soaked in the fecal fluid simulant for the full 30 minutes, centrifuge the holder with the soaked superabsorbent in it. The holder is placed in a cylinder with the spacers and screen as shown in FIG. 1. Set the centrifuge at 1250 rpm. The sample spins for 3 minutes.

Remove the holder containing the superabsorbent from the centrifuge cylinder. Weigh the holder with the swollen superabsorbent and cover screen. Record the weight on the data sheet. Also record the name of the superabsorbent sample. Make note of anything that looks irregular such as dry superabsorbent remaining in the holder, etc.

To find the CRC value, use the following equation:

[W3-(W1+W2)]/W2

Where:

W1=Mass of the holder and screen alone

W2=Mass of the superabsorbent

W3=Mass of the swollen superabsorbent, holder and screen

The formula gives the unitless CRC value associated with the particular superabsorbent tested.

While the embodiments of the invention described herein are presently preferred, various modifications and improvements can be made without departing from the spirit and scope of the invention. The scope of the invention is indicated by the appended claims, and all changes that fall within the meaning and range of equivalents are intended to be embraced therein.

We claim:

1. A heterogeneous absorbent core including a front region, a crotch region, a back region, a front edge, a back edge and two side edges, the core comprising:
a first zone, including a first superabsorbent material having a saline AUL at 0.3 psi of at least about 15 grams/gram; and
a second zone, including a second superabsorbent material different from the first having a fecal fluid AUL at 0.3 psi of at least about 15 grams/gram;
wherein the zones are positioned so that the first zone is closer to the front edge and the second zone is closer to the back edge.

2. The heterogeneous absorbent core of claim 1, wherein the first zone comprises at least part of the front region.

3. The heterogeneous absorbent core of claim 1, wherein the first zone comprises at least part of the crotch region.

4. The heterogeneous absorbent core of claim 2, wherein the first zone further comprises at least part of the crotch region.

5. The heterogeneous absorbent core of claim 1, wherein the second zone comprises at least part of the back region.

6. The heterogeneous absorbent core of claim 1, wherein the second zone comprises at least part of the crotch region.

7. The heterogeneous absorbent core of claim 5, wherein the second zone further comprises at least part of the crotch region.

8. The heterogeneous absorbent core of claim 1, wherein the first and second zones are adjacent to each other.

9. The heterogeneous absorbent core of claim 1, further comprising a third zone between the first and second zones.

10. The heterogeneous absorbent core of claim 9, wherein the third zone comprises the first superabsorbent material and the second superabsorbent material.

11. The heterogeneous absorbent core of claim 1, wherein the first superabsorbent material has a saline AUL of at least about 25 grams/gram.

12. The heterogeneous absorbent core of claim 1, wherein the first superabsorbent material has a saline AUL of at least about 30 grams/gram.

13. The heterogeneous absorbent core of claim 1, wherein the second superabsorbent material has a fecal fluid AUL of at least about 20 grams/gram.

14. The heterogeneous absorbent core of claim 1, wherein the second superabsorbent material has a fecal fluid AUL of at least about 25 grams/gram.

15. The heterogeneous absorbent core of claim 1, further comprising a relatively depressed region surrounded by a relatively raised region in at least the back region of the absorbent core.

16. The heterogeneous absorbent core of claim 15, wherein the relatively depressed region and relatively raised region further extend into the crotch region of the absorbent core.

17. The heterogeneous absorbent core of claim 16, wherein the relatively depressed region and relatively raised region further extend into the front region of the absorbent core.

18. An absorbent article including a liquid-permeable bodyside liner, an outer cover and a heterogeneous absorbent core between them;
the absorbent core including a front region, a crotch region, a back region, a first zone and a second zone;
the first zone comprising at least part of the front region and including a first superabsorbent material having a saline AUL of at least about 15 grams/gram;
the second zone comprising at least part of the back region and including a second superabsorbent material, different from the first, having a saline AUL of at least about 15 grams/gram and a fecal fluid AUL of at least about 15 grams/gram.

19. The absorbent article of claim 18, wherein the first superabsorbent material has a saline AUL of at least about 25 grams/gram.

20. The absorbent article of claim 18, wherein the second superabsorbent material has a saline AUL of at least about 25 grams/gram and a fecal fluid AUL of at least about 20 grams/gram.

21. The absorbent article of claim 18, wherein the first and second zones each comprise a fiber matrix, the first superabsorbent material is dispersed in the fiber matrix in the first zone, and the second superabsorbent material is dispersed in the fiber matrix in the second zone.

22. The absorbent article of claim 18, wherein at least one of the first and second zones comprises a film which includes at least one of the first and second superabsorbent materials.

23. The absorbent article of claim 18, wherein at least one of the first and second zones comprises a foam which includes at least one of the first and second superabsorbent materials.

24. The absorbent article of claim 18, wherein the first superabsorbent material comprises a polymer selected from alkali metal salts of polyacrylic acid, ammonium salts of polyacrylic acid, alkali metal salts of polymethacrylic acid, polyacrylates, ammonium salts of polymethacrylic acid, polyacrylamides, hydrolyzed maleic anhydride copolymers with vinyl ethers, hydrolyzed maleic anhydride copolymers with alpha-olefins, polyacrylates, polymers and copolymers of vinyl sulfonic acid, hydrolyzed acrylonitrile-grafted starch, partially neutralized acrylic acid-grafted starch, carboxymethyl cellulose, multicomponent superabsorbent polymers, and combinations thereof.

25. The absorbent article of claim 18, wherein the second superabsorbent material comprises a polymer selected from the group consisting of multicomponent superabsorbent polymers, polyacrylates, polyvinyl amines, polyacrylic acids, and combinations thereof.

26. The absorbent article of claim 18, selected from the group consisting of a diaper, a training pant, swim wear, and an adult incontinence garment.

27. The absorbent article of claim 18, further comprising a relatively depressed region and a relatively raised region in at least part of the absorbent core.

28. An absorbent article including a liquid-permeable bodyside liner, an outer cover and a heterogeneous absorbent core between them,
the absorbent core including a front region, a crotch region, a back region, a first zone and a second zone,
the first zone comprising at least part of the front or crotch region and including a first superabsorbent material having a saline AUL of at least about 15 grams/gram;
the second zone comprising at least part of the back or crotch region and including a second superabsorbent having a fecal fluid AUL of at least about 15 grams/gram;
wherein the first and second superabsorbent materials are present in the first and second zones in substantially different ratios which differ by at least about 10%.

29. The absorbent article of claim 28, wherein the first and second superabsorbent materials are present in the first and second zones in substantially different ratios which differ by at least about 25%.

30. The absorbent article of claim 28, wherein the first and second superabsorbent materials are present in the first and second zones in substantially different ratios which differ by at least about 50%.

31. The absorbent article of claim 28, selected from the group consisting of a diaper, a training pant, swim wear, and an adult incontinence garment.

32. The absorbent core of claim 28, further comprising a relatively depressed region surrounded by a relatively raised region in at least part of the absorbent core.

33. A bi-functional absorbent core including at least one layer having a front region, a crotch region and a back region, each region having a length, the core comprising:
a first superabsorbent material having a saline AUL of at least about 15 grams/gram, present along the length of the crotch region and in the front region; and
a second superabsorbent material different from the first, having a fecal fluid AUL of at least about 15 grams/gram, present along the length of the crotch region and in the back region.

34. The bi-functional absorbent core of claim 33, wherein the first superabsorbent material is also present in the back region.

35. The bi-functional absorbent core of claim 33, wherein the second superabsorbent material is also present in the front region.

36. The bi-functional absorbent core of claim 34, wherein the second superabsorbent material is also present in the front region.

37. The bi-functional absorbent core of claim 33, comprising a first layer including the first superabsorbent material and a second layer including the second superabsorbent material.

38. The bi-functional absorbent core of claim 37, further comprising a third layer.

39. The bi-functional absorbent core of claim 33, comprising a first layer which includes the first superabsorbent material and the second superabsorbent material.

40. The bi-functional absorbent core of claim 33, wherein the first superabsorbent material has a saline AUL of at least about 25 grams/gram.

41. The bi-functional absorbent core of claim 33, wherein the first superabsorbent material has a saline AUL of at least about 30 grams/gram.

42. The bi-functional absorbent core of claim 33, wherein the second superabsorbent material has a fecal fluid AUL of at least about 20 grams/gram.

43. The bi-functional absorbent core of claim 33, wherein the second superabsorbent material has a fecal fluid AUL of at least about 25 grams/gram.

44. The bi-functional absorbent core of claim 33, wherein the second superabsorbent material has a saline AUL of at least about 15 grams/gram.

45. An absorbent article including a liquid-permeable bodyside liner, an outer cover and a bi-functional absorbent core between them;
the absorbent core including at least one layer having a front region, a crotch region and a back region;
the crotch region comprising a first superabsorbent material having a saline AUL of at least about 15 grams/gram and a second superabsorbent material having a fecal fluid AUL of at least about 15 grams/gram;
the front region comprising the first superabsorbent material;
the back region comprising the second superabsorbent material.

46. The absorbent article of claim 45, wherein the front region further comprises the second superabsorbent material.

47. The absorbent article of claim 45, wherein the back region further comprises the first superabsorbent material.

48. The absorbent article of claim 46, wherein the back region further comprises the first superabsorbent material.

49. The absorbent article of claim 45, wherein the absorbent core comprises two layers, the first superabsorbent material is present in the first layer, and the second superabsorbent material is present in the second layer.

50. The absorbent article of claim 49, wherein the first layer is positioned to receive fluid before the second layer.

51. The absorbent article of claim 49, wherein the second layer is positioned to receive fluid before the first layer.

52. The absorbent article of claim 45, wherein the at least one layer further comprises a fiber matrix, and at least one of the first and second superabsorbent materials is dispersed in the matrix.

53. The absorbent article of claim 49, wherein each layer further comprises a fiber matrix, the first superabsorbent material is dispersed in the fiber matrix in the first layer, and the second superabsorbent material is dispersed in the fiber matrix in the second layer.

54. The absorbent article of claim 45, wherein the at least one layer comprises a film which includes at least one of the first and second superabsorbent materials.

55. The absorbent article of claim 45, wherein the at least one layer comprises a bi-component fibrous web including the first and second superabsorbent polymers.

56. The absorbent article of claim 45, wherein the at least one layer comprises a foam which includes at least one of the first and second superabsorbent materials.

57. The absorbent article of claim 45, wherein the first superabsorbent material comprises a polymer selected from alkali metal salts of polyacrylic acid, ammonium salts of polyacrylic acid, alkali metal salts of polymethacrylic acid, polyacrylates, ammonium salts of polymethacrylic acid, polyacrylamides, polyvinylalcohol, polyphosphazene, polyvinyl ethers, hydrolyzed maleic anhydride copolymers with vinyl ethers, hydrolyzed maleic anhydride copolymers with alpha-olefins, polyacrylates, polyvinyl pyrrolidone, polyvinyl pyridine, polyvinyl morpholinone, polymers and copolymers of vinyl sulfonic acid, hydrolyzed acrylonitrile-grafted starch, acrylic acid-grafted starch, methyl cellulose, chitosan, carboxymethyl cellulose, hydroxypropyl cellulose, natural gums, and combinations thereof.

58. The absorbent article of claim 45, wherein the second superabsorbent material comprises a polymer selected from multicomponent superabsorbent polymers, polyacrylates, polyvinyl amines, polyacrylic acids, and combinations thereof.

59. The absorbent article of claim 45, selected from the group consisting of a diaper, a training pant, swim wear, and an adult incontinence garment.

60. An absorbent article including a liquid-permeable bodyside liner, an outer cover and a bi-functional absorbent core between them,
the absorbent core comprising first and second layers,
the first layer including a first superabsorbent material having a saline AUL of at least about 20;
the second layer including a second superabsorbent material having a saline AUL of at least about 20 and a fecal fluid AUL of at least about 15.

61. The absorbent article of claim 60, selected from the group consisting of a diaper, a training pant, swim wear, and an adult incontinence garment.

62. The heterogeneous absorbent core of claim 1, wherein the second zone further comprises a matrix material having an open structure.

63. The absorbent article of claim 18, wherein the second zone further comprises a matrix material having an open structure.

64. The absorbent article of claim 28, wherein the second zone further comprises a matrix material having an open structure.

65. The bi-functional absorbent core of claim 33, wherein at least one of the crotch and back regions further comprises a matrix material having an open structure.

66. The absorbent article of claim 45, wherein at least one of the crotch and back regions further comprises a matrix material having an open structure.

67. The absorbent article of claim 60, wherein at least part of the first layer comprises a matrix material having an open structure.

68. A method for preparing a fecal fluid simulant, comprising the steps of:
determining the fecal fluid AUL of a fecal fluid sample;
analyzing the fecal fluid sample to determine its composition;
determining the effect of fecal fluid components on the fecal fluid AUL; and
preparing a fecal fluid simulant having about the same fecal fluid AUL as the fecal fluid sample, using simulated fecal fluid components.

69. A fecal fluid simulant comprising an aqueous mixture of sodium chloride, dextran, and egg white.

70. The fecal fluid simulant of claim 69, having a fecal fluid AUL of about 11.2 to about 17.2 grams per gram.

71. The fecal fluid simulant of claim 69, having a fecal fluid AUL of about 5.8 to about 17.2 grams per gram.

72. The fecal fluid simulant of claim 69, having a fecal fluid AUL of about 5.8 to about 8.4 grams per gram.
